(19) United States
(12) Patent Application Publication
(10) Pub. No.: US 2020/0323433 A1
(43) Pub. Date: Oct. 15, 2020

(54) CAPSULE ENDOSCOPE AND CONTROL METHOD THEREOF

(71) Applicant: ANKON TECHNOLOGIES CO., LTD, Wuhan (CN)

(72) Inventors: Fanhua MING, Wuhan (CN); Hangyu PENG, WUHAN (CN); RONG WANG, WUHAN (CN); Bo FENG, Wuhan (CN); Nianqi ZHOU, Wuhan (CN)

(73) Assignee: ANKON TECHNOLOGIES CO., LTD, Wuhan (CN)

(21) Appl. No.: 16/843,373

(22) Filed: Apr. 8, 2020

(30) Foreign Application Priority Data
Apr. 12, 2019 (CN) 201910292174.2

(51) Int. Cl. A61B 5/00; A61B 1/04; A61B 1/06; A61B 1/00

(52) U.S. Cl. CPC A61B 5/0084; A61B 1/041; A61B 1/0661; A61B 1/0638; A61B 1/00158; A61B 1/00006; A61B 1/00009; A61B 5/0086; A61B 1/00016; A61B 1/00195

(57) ABSTRACT

The present invention provides a capsule endoscope and a control method thereof. The capsule endoscope includes an enclosure, a photographing device and a light source device housed in the enclosure. The light source device illuminates inner surface of digestive tract with illumination light, and the photographing device captures images of the digestive tract based on the illumination light. The enclosure comprises a main enclosure and an optical dome enclosed at one end of the main enclosure. The optical dome is disposed at the front end of the light source device. The light source device comprises a first light source module and a second light source module. The wavelength ranges of lights emitted from the first light source module and the second light source module are different, and the first light source module and the second light source module are arranged with a space around the photographing device.

### CROSS-REFERENCE OF RELATED APPLICATIONS

The application claims priority to Chinese Patent Application No. 201910292174.2 filed on Apr. 12, 2019, the contents of which are incorporated by reference herein.

### FIELD OF INVENTION

The present invention relates to a medical device, and more particularly to a capsule endoscope and a control method thereof.

### BACKGROUND

In recent years, in the field of endoscopes, a swallowable capsule endoscope provided with photographing function and wireless communication function has been proposed. The capsule endoscope is intended to take images of the digestive tract of a subject at an interval of, for example, 0.5 seconds as moving along with the peristalsis of stomach and small intestine after being swallowed by the subject until being naturally expelled.

In addition, while the capsule endoscope is moving inside the digestive tract, images taken by are sequentially transmitted from the capsule endoscope to an external receiving device via a wireless communication module. The receiving device has a function of wirelessly communicating with the capsule endoscope and a storage function, and receives images wirelessly transmitted from the capsule endoscope, and sequentially stores the received images in a storage module. By carrying the receiving device, the subject is free to move from swallowing the capsule endoscope until naturally expelling it.

After the capsule endoscope is naturally expelled from the subject, a physician or nurse transfers the image group stored in the storage module of the receiving device to an image display device, and makes the images captured inside the subject (specifically, images of the digestive tract) displayed on the image display device. The physician or nurse can diagnose the subject by observing the images displayed on the image display device.

However, the existing capsule endoscope only takes images of epidermis of the digestive tract for subsequent diagnosis. Many early lesions often appear on the surface of mucosal cells. Generally, the ordinary camera of traditional capsule endoscope fails to capture this fine structure.

In order to improve the display clarity of pit pattern at the mucosal surface, and at the same time to clearly observe the pattern and structure of capillaries, to increase the detection rate of flat and tiny lesions in intestinal tract, a capsule endoscope with more image display effects is required. So, it is necessary to provide an improved capsule endoscope and to solve the said technical issues.

### SUMMARY OF THE INVENTION

The present invention provides a capsule endoscope to improve the diagnosis rate of lesions.

The present invention further provides a control method of the capsule endoscope to improve the diagnosis rate of lesions.

To achieve the objects, the present invention provides a capsule endoscope, which is introduced into a subject and captures images of digestive tract of the subject, comprising an enclosure and a photographing device and a light source device housed in the enclosure. The light source device illuminates inner surface of the digestive tract with illumination light, and the photographing device captures images of digestive tract based on the illumination light. The enclosure comprises a main enclosure and an optical dome enclosed at one end of the main enclosure. The optical dome is disposed at the front end of the photographing device and the light source device. The light source device comprises a first light source module and a second light source module, wherein the wavelength ranges of lights emitted from the first light source module and the second light source module are different, and the first light source module and the second light source module are arranged with a space around the photographing device.

In accordance with an embodiment, the first light source module comprise at least two standard polychromatic light emitting elements and the second light source module comprises at least two narrow-band light emitting elements. The standard polychromatic light emitting elements and the narrow-band light emitting elements are evenly arranged around the photographing device.

In accordance with an embodiment, the light source device further comprises a third light source module. The first light source module, the second light source module, and the third light source module are arranged with equal spacing around the photographing device. The wavelength range of light emitted from the third light source module is different from that of the first light source module and the second light source module.

In accordance with an embodiment, the first light source module comprises 3-6 standard polychromatic light emitting elements, the second light source module comprises 3-6 narrow-band light emitting elements, and the third light source module comprises 3-6 monochromatic light emitting elements. The standard polychromatic light emitting elements, the narrow-band light emitting elements and the monochromatic light emitting elements come with the same quantity and are evenly arranged around the photographing device.

In accordance with an embodiment, the monochromatic light emitting elements are all configured as infrared light emitting elements, and the wavelength of infrared light emitted by the infrared light emitting elements is in a range of 780 nm to 2526 nm.

In accordance with an embodiment, an annular circuit board is disposed in the enclosure, and the first light source module, the second light source module and the third light source module are arranged with equal spacing on the annular circuit board.

In accordance with an embodiment, the annular circuit board has a through hole, and the annular circuit board is fixed on the photographing device, at least part of the photographing device passes through the through hole.

In accordance with an embodiment, the photographing device comprises an optical lens and an image sensor. The optical lens is disposed through the through hole, and the image sensor is fixed to a side of the optical lens away from the optical dome.

In accordance with an embodiment, the capsule endoscope further comprises an image processing and control device that connects to the photographing device and the light source device. The image processing and control device controls the first light source module and/or the second light source module to be turned on and off based on the images taken by the photographing device.

In accordance with an embodiment, the image processing and control device comprises an image identification and calculation unit that processes the images taken by the photographing device and determines if the images are abnormal. When an image is determined as abnormal, the image processing and control device controls the first light source module to be turned off and the second light source module to be turned on.

The present invention also relates to a method of controlling the capsule endoscope, comprising the following steps:

turn on the first light source module;

capture an image of the digestive tract of the subject;

process the captured image and determine if the image is abnormal;

when the image is abnormal, turn off the first light source module and turn on the second light source module, and capture an image of the digestive tract of the subject again.

In accordance with an embodiment, the light source device further comprises a third light source module. The first light source module, the second light source module, and the third light source module are arranged with equal spacing around the photographing device. The wavelength range of light emitted from the third light source module is different from that of the first light source module and the second light source module. The control method further comprises the following steps:

turn off the second light source module and turn on the third light source module, and capture an image of the digestive tract of the subject.

transmit the captured image wirelessly to outside of the subject.

In accordance with an embodiment, an image identification algorithm is used to process the captured image and determine if the image is abnormal.

Compared with the prior art, the present invention provides a capsule endoscope comprising at least two different light source devices, so that the capsule endoscope can not only capture clear images of ordinary lesions, but it can also capture images of different levels of mucosal patterns, so as to detect deep lesions and effectively improve the diagnosis rate of lesions.

### BRIEF DESCRIPTION OF THE DRAWINGS

FIG. 1 is a sectional view of the capsule endoscope in a preferred embodiment of the present invention.

FIG. 2 is a sectional view of the capsule endoscope along the line A-A shown in FIG. 1.

FIG. 3 is a system block diagram of the capsule endoscope in FIG. 1.

FIG. 4 is a control flowchart of the capsule endoscope in FIG. 1.

### DETAILED DESCRIPTION

The present invention can be described in detail below with reference to the accompanying drawings and preferred embodiments. However, the embodiments are not intended to limit the invention, and the structural, method, or functional changes made by those skilled in the art in accordance with the embodiments are included in the scope of the present invention.

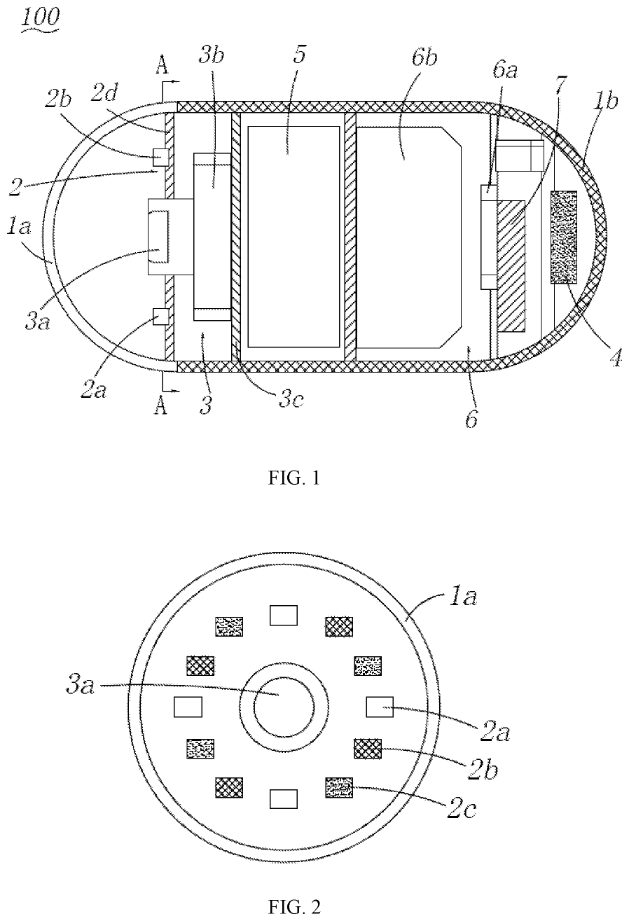

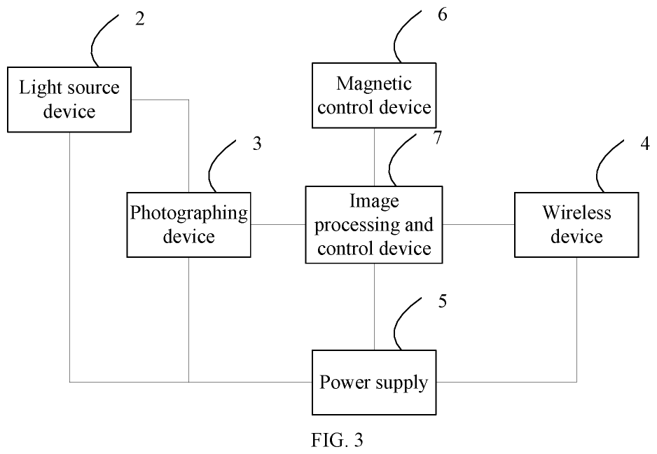

Referring to FIG. 1 to FIG. 3, according to a preferred embodiment of the present invention, the capsule endoscope is introduced into a subject and captures images of digestive tract of the subject. The capsule endoscope 100 comprises an enclosure and a light source device 2, a photographing device 3, a wireless device 4, a power supply 5, a magnetic control device 6 and an image processing and control device 7 housed in the enclosure. The enclosure comprises a main enclosure 1b and an optical dome 1a enclosed at one end of the main enclosure 1b. The optical dome 1a is disposed at the front end of the photographing device 3 and the light source device 2. The main enclosure 1b and the optical dome 1a roughly form a capsule shape. The main enclosure 1b is generally opaque, and the optical dome 1a is a roughly transparent hemisphere with high light transmittance.

The light source device 2 is used for illumination and comprises a first light source module 2a and a second light source module 2b. The wavelength ranges of lights emitted from the first light source module 2a and the second light source module 2b are different, and the first light source module 2a and the second light source module 2b are arranged with a space around the photographing device 3. In the embodiment, the first light source module 2a can be an ordinary light emitting device disposed at the front end of the capsule for capturing images with a conventional lens, which can emit visible light to illuminate the inner wall of the digestive tract through the transparent optical dome 1a. The second light source module 2b can be a narrow-band light-emitting device with a special filtering function used in NBI (Narrow Band Imaging) technology, which can filter ordinary spectrum and retain only narrow-band light waves, thus providing an effective light source for NBI implementation. In the embodiment, the light source device 2 further comprises a third light source module 2c. The first light source module 2a, the second light source module 2b, and the third light source module 2c are arranged with equal spacing around the photographing device 3. The wavelength range of light emitted from the third light source module 2c is different from that of the first light source module 2a and the second light source module 2b. The third light source module 2c can be a special light-emitting device that generates only monochromatic light, such as infrared light, to obtain deep skin tissue information. In addition, it can also be any other type of light-emitting device.

The first light source module 2a preferably comprises at least two standard polychromatic light emitting elements for emitting visible light, such as a conventional white light LED which emits light for illuminating the field of view of the photographing device 3, and can irradiate the emitted visible light on the inner wall of the digestive tract through the transparent optical dome 1a. The second light source module 2b is a light-emitting device that is, for example, a light-emitting component such as a traditional LED with coating, or provided with a narrow-band filter. The second light source module 2b is a light-emitting device with a special filtering function, which comprises at least two narrow-band light emitting elements and can emit a narrow-band spectrum having a specific wavelength to illuminate the field of view of the photographing device 3 using NBI technology. The third light source module 2c only generates monochromatic light, such as infrared light, to obtain deep skin tissue information. The third light source module 2c comprises at least two monochromatic light emitting elements, such as infrared light emitting elements, and preferably emits near-infrared light having a wavelength ranging from 780 to 2526 nm, which can penetrate tissue and directly act on the submucosal blood vessels, lymphatic vessels, nerve endings and other deep tissues. Among them, at least two standard polychromatic light emitting elements, at least two narrow-band light emitting elements, and at least two monochromatic light emitting elements can be evenly and alternately arranged around the photographing device 3. The three light source modules are only a preferred embodiment of the present invention, and two or more light source modules can also be provided according to actual needs. Multi-light source design can ensure adaptability to the imaging of the digestive tract of different levels and scenarios, and improve the accuracy of diagnosis.

An annular circuit board 2d matching the shape of inner wall of the enclosure is disposed in the enclosure. The annular circuit board 2d can be installed inside the main enclosure 1b or inside the optical dome 1a. At least two standard polychromatic light emitting elements, at least two narrow-band light emitting elements and at least two monochromatic light emitting elements are arranged on the annular circuit board 2d, and are electrically connected to the annular circuit board 2d. These light emitting elements are evenly and alternately arranged on the circumference of the annular circuit board 2d, that is, every two adjacent light emitting elements are different, so that the circumferential distance between the same light emitting elements is the same, and the emitted light is more uniform.

In the embodiment, the number of each type of light emitting element is 3-6. Preferably, the number of each type of light emitting element is 4, and the three light source modules come with a total of 12 light emitting elements. The 12 light emitting elements are evenly arranged on the circumference of the annular circuit board 2d in order, which can meet the illuminating and imaging effect.

Further, the photographing device 3 is configured to capture images inside the digestive tract of the subject, and can capture the targets in a field of view illuminated by the light source device 2. The annular circuit board 2d comprises a through hole through which a part of the photographing device 3 passes, so that the light source 2 cannot be blocked by the lens as the light emitting elements surrounds the photographing device 3. In addition, the photographing device 3 has a certain height along the axial direction of the enclosure and a part of the photographing device 3 passes through the through hole, making the structure inside the enclosure more compact, and thereby reducing the overall size of the capsule endoscope.

The photographing device 3 comprises an optical lens 3a, an image sensor 3b, and a photographing circuit board 3c. The optical lens 3a is used for forming images taken in the subject on a light receiving surface of the photographing device 3. The image sensor 3b is configured to be a solid-state image sensor such as CMOS for capturing images of the subject. The photographing circuit board 3c has a circuit formed to achieve the function of the photographing device. The photographing circuit board 3c is electrically connected to the annular circuit board 2d, and the annular circuit board 2d is fixed on the photographing device 3. In an example, the annular circuit board 2d can be snapped on the photographing device 3. The image sensor 3b is disposed at the rear of the optical lens 3a. The side near the optical dome 1a is called front and the other side is called rear. The image sensor 3b is adhesively bonded to the optical lens 3a and has the function of converting optical signals taken by the optical lens 3a into electrical signals, and transmitting the image signals to the image processing and control device 7 for image display processing and system control.

The image processing and control device 7 is not only used for compressing and converting the digital signals of images taken by the photographing device 3, but also for controlling the light source device 2 to be turned on and off according to the image information. The multi-light source device can be controlled by the image processing and control device 7 according to different needs in different situations, so as to obtain more complete image information of the digestive tract and improve diagnostic accuracy. Generally, ordinary light sources are used for photographing, and when a lesion is found, it is determined whether to turn on narrow-band light and infrared light. For example, an image recognition algorithm can be used for automatic identification and automatically turning on light source, that is, determining whether the light source should be controlled according to whether the image is abnormal.

The wireless device 4 is mainly used for wirelessly transmitting and receiving image data. That is, The wireless device 4 transmits the image data collected and processed by the image processing and control device 7 to an external data recorder, and at the same time, receives external commands.

The magnetic control device 6 comprises a magnetic control sensor 6a and a magnetic dipole 6b with magnetic control function, which can be controlled by external magnetic field for adjusting the posture of the capsule endoscope in the subject, and controlling the capsule endoscope to rotate horizontally and vertically and move forwards and backwards. Through the magnetic control device 6, the capsule endoscope can be actively controlled, and the position and orientation of the capsule endoscope in the digestive tract can be provided in real time, so that a guarantee for effective control of the capsule endoscope in examination is ensured.

The power supply 5 is disposed between the photographing device 3 and the magnetic control device 6 and is used to supply power to the capsule endoscope for all functional activities in the digestive tract. The power supply 5 can be, for example, a silver oxide button battery.

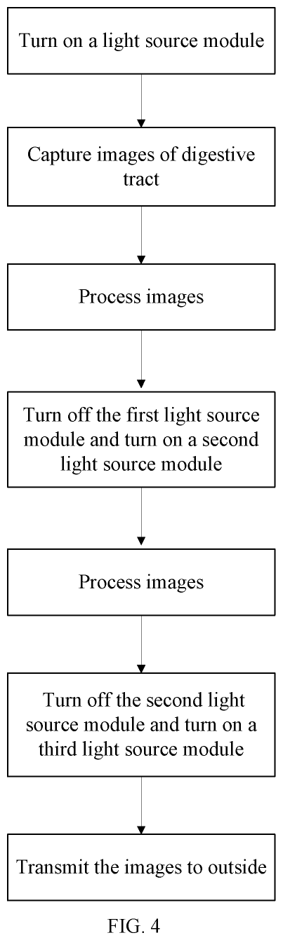

The following describes in detail the function of the photographing device 3 and the control flow of the three light source modules in the embodiment. As shown in FIG. 4, when the capsule endoscope 1 enters the digestive tract of an subject, the image processing and control device 7 turns on the first light source module 2a to illuminate the digestive tract, the photographing device 3 takes images of the digestive tract, and after being processed by the image processing and control device 7, the images are transmitted to the outside of the subject through the wireless device 8.

When a physician or nurse finds a suspected lesion at a certain region according to the captured images, and needs to obtain the deeper information of the region, or when the external processor receives an image transmitted from the wireless device 8, and determines that the image is abnormal after processing the image according to a pre-stored algorithm, the image processing and control device 7 can be ordered to turn off the first light source module 2a and turn on the second light source module 2b to emit narrow-band light with a special filtering function, so that the photographing device 3 can perform narrow-band imaging. Preferably, the image processing and control device 7 comprises an image identification and calculation unit which automatically identifies abnormal images and automatically turns off the first light source module 2a and turns on the second light source module 2b.

When deeper information on blood vessels and nerve endings is needed, or the image captured when the second light source module 2b is turned on is identified as abnormal, the image processing and control device 7 turns off the narrow-band light emitting elements and turns on the special third light source module 2c, and transmits the images to outside of the patient via the wireless transmission device 8. In addition, the wireless transmission of images described above can be conducted after each photographing is completed, or can be conducted at a time after all photographing tasks are completed. The processing flow with a control function can ensure that the images taken by the conventional photographing device and the images taken by that with special light source device completely coincide, which is conducive to determining the location of a lesion and more accurately analyzing the condition of the lesion.

The present invention provides such a multi-light source controllable capsule endoscope, which not only has a function of taking images under visible light to clearly display the normal image effect inside digestive tract, but also can obtain images taken under a narrow-band light emitting element to observe the information of surface layer and lower layer of mucosal glands, and can obtain image information of deeper layer via near-infrared light emitted by a special monochromatic light emitting elements. This helps the physician to predict the type of pathological tissue and the depth of invasion of early cancer, so as to provides guide for treatment. It is of great significance for the diagnosis of early gastrointestinal cancer.

It should be understood that, although the specification is described in terms of embodiments, not every embodiment merely includes an independent technical solution. Those skilled in the art should have the specification as a whole, and the technical solutions in each embodiment may also be combined as appropriate to form other embodiments that can be understood by those skilled in the art.

The present invention by no means is limited to the preferred embodiments described above. On the contrary, many modifications and variations are possible within the scope of the appended claims.

What is claimed is:

1. A capsule endoscope, which is introduced into a subject and captures images of digestive tract of the subject, comprising:
an enclosure, a photographing device and a light source device housed in the enclosure, wherein
the light source device illuminates inner surface of the digestive tract with illumination light, and the photographing device takes images of the digestive tract based on the illumination light;
the enclosure comprises a main enclosure and an optical dome enclosed at one end of the main enclosure, wherein the optical dome is disposed at the front end of the photographing device and the light source device; and wherein
the light source device comprises a first light source module and a second light source module, wherein the lights emitted from the first light source module and the second light source module are different in wavelength range, and the first light source module and the second light source module are arranged with a space around the photographing device.

2. The capsule endoscope of claim 1, wherein the first light source module comprises at least two standard polychromatic light emitting elements and the second light source module comprises at least two narrow-band light emitting elements, wherein the standard polychromatic light emitting elements and the narrow-band light emitting elements are evenly arranged around the photographing device.

3. The capsule endoscope of claim 1, wherein the light source device further comprises a third light source module, wherein the first light source module, the second light source module and the third light source module are arranged with equal spacing around the photographing device, and the wavelength range of light emitted from the third light source module is different from that of the first light source module and the second light source module.

4. The capsule endoscope of claim 3, wherein the first light source module comprises 3-6 standard polychromatic light emitting elements, the second light source module comprises 3-6 narrow-band light emitting elements, and the third light source module comprises 3-6 monochromatic light emitting elements, wherein the standard polychromatic light emitting elements, the narrow-band light emitting elements and the monochromatic light emitting elements come with the same quantity and are evenly and alternately arranged around the photographing device.

5. The capsule endoscope of claim 4, wherein the monochromatic light emitting elements are all configured as infrared light emitting elements, and the wavelength of infrared light emitted by the infrared light emitting elements is in a range of 780 nm to 2526 nm.

6. The capsule endoscope of claim 3, wherein an annular circuit board is disposed in the enclosure, and the first light source module, the second light source module and the third light source module are arranged with equal spacing on the annular circuit board.

7. The capsule endoscope of claim 6, wherein the annular circuit board comprises a through hole, and the annular circuit board is fixed on the photographing device, at least part of the photographing device passes through the through hole.

8. The capsule endoscope of claim 7, wherein the photographing device comprises an optical lens and an image sensor, wherein the optical lens is disposed through the through hole, and the image sensor is fixed to a side of the optical lens away from the optical dome.

9. The capsule endoscope of claim 1, further comprising an image processing and control device that connects to the photographing device and the light source device, wherein the image processing and control device controls the first light source module and/or the second light source module to be turned on and off based on the images taken by the photographing device.

10. The capsule endoscope of claim 9, wherein the image processing and control device comprises an image identification and calculation unit that processes the images taken by the photographing device and determines if the images are abnormal, and when an image is determined as abnormal, the image processing and control device controls the first light source module to be turned off and the second light source module to be turned on.

11. A control method of the capsule endoscope of claim 1, comprising the following steps:
turn on the first light source module;
capture an image of the digestive tract of the subject;
process the captured image and determine if the image is abnormal;
when the image is abnormal, turn off the first light source module and turn on the second light source module, and capture an image of the digestive tract of the subject again.

12. The control method of claim 11, wherein the light source device further comprises a third light source module, the first light source module, the second light source module and the third light source module are arranged with equal spacing around the photographing device, and the wavelength range of light emitted from the third light source module is different from that of the first light source module and the second light source module, further comprising the following steps:
turn off the second light source module and turn on the third light source module, and capture an image of the digestive tract of the subject;
transmit the captured image wirelessly to outside of the subject.

13. The control method of claim 11, wherein an image identification algorithm is used to process the captured images and determine if the images are abnormal.
